SEQUENCE LISTING (6 sequences)

SEQ ID NO: 1 - DNA, 1251 bp, Nicotiana tabacum
  CDS (22)..(1089)
gcatccctctctcttctccacatggcatctttagctctacctggaagctttttagctaccagcaaacctagtccttgtagatatgctgccggagactcaattgtaagatgtaatgtggcagaaccattaagttttaacaaggagaatgggagatcaaacatgcctcttcagattaatggcgacacttcatttaacaatctttggaacgctaatcaagtaagaagatttccagttccacatgctcagattgatactagactccgcattttctccggcactgccaatcctgcactttctcaggaaatagcttgctacatgggtttggaacttggaaagataatgataaaacgttttgctgatggcgaaatctatgtccagttacaagagagtgttaggggttgtgatgtatatcttgtccaacctacgtgtctcctgctaatgaaatctgatggactcttgataatgattgatgcttgccgtagagcctcagccaaaaatattactgcagtgattccgtactttgggtatgcccgtgctgatcgtaagactcaaggtcgtgaatcgattgctgccaaacttgtagcaaacctgattacagaagctggtgcagatcgagttcttgcttgtgatcttcattctggccagtcaatgggttactttgatattccagtggatcatgtacatggccagcctgtcatacttgattaccttgccagcaagactatctgctctgatgatctagttgtggtatctccggatgttggtggggttgcaagggcaagagcttttgccaaaaagttatctgatgcacctctagctattgtggataaaaggcgtcatgggcacaatgttgctgaggtaatgaatttgattggcgatgttaggggaaaagtggcagttatggttgatgacatgattgatacggctggtactattgcaaaaggagctgcccttttacatcaaggagccagggaagtttatgcatgcaccactcatgcagttttcagggcggctttgagccttactccggattgggaattgattagtccttcctgttcattgcttgttttgacactgttgtctggaacttgaagcagtatgttgatcagatccaccaaaattttcgtccagtaacaaagagagcattttctgttgtacaaatattttgtgcggaatgttacgttgtaaatctttcaatcagtgacttggatccatgtagtagatgagttttttgattaatatgaagtttac

SEQ ID NO: 2 - protein, 356 aa, Nicotiana tabacum
MASLALPGSFLATSKPSPCRYAAGDSIVRCNVAEPLSFNKENGRSNMPLQINGDTSFNNLWNANQVRRFPVPHAQIDTRLRIFSGTANPALSQEIACYMGLELGKIMIKRFADGEIYVQLQESVRGCDVYLVQPTCLLLMKSDGLLIMIDACRRASAKNITAVIPYFGYARADRKTQGRESIAAKLVANLITEAGADRVLACDLHSGQSMGYFDIPVDHVHGQPVILDYLASKTICSDDLVVVSPDVGGVARARAFAKKLSDAPLAIVDKRRHGHNVAEVMNLIGDVRGKVAVMVDDMIDTAGTIAKGAALLHQGAREVYACTTHAVFRAALSLTPDWELISPSCSLLVLTLLSGT

SEQ ID NO: 3 - DNA, 942 bp, Arabidopsis thaliana
  CDS (1)..(939)
atggtcttgaagttgttctctggtactgcaaatccagcacttgctcaggaaattgcttggtatatgggcttggatcttggcaaggttaatattaagaggtttgctgatggagagatctatgttcagctacaagagagcgttaggggatgtgacgtctatttggtgcagcctacttgcactcccactaatgagaatctcatggagcttttgattatggtagatgcttgccgaagagcatcagctaagaaagttacagctgtgattccgtattttggatatgcaagagctgacaggaagacacaagggcgtgaatccattgctgccaaattggttgcaaatcttatcactgaagctggtgcagatcgagttcttgcttgtgatcttcattcaggacagtcgatgggttattttgacattccagtcgaccatgtgtactgccagccggtgatacttgattatcttgctagcaagtcaattccctcagaggatttggtagtggtttctcctgatgttggtggagtagccagggcccgcgcttttgcaaagaaattatcagatgcaccacttgccattgtcgataaaaggcgttctggacacaatgttgctgaggtcatgaacctaattggtgatgtaagagggaaggtggcaataatggtggatgatatgattgatactgctggaaccattgtaaaaggagcagctttgttacaccaggaaggtgctcgggaggtatatgcgtgctgcacacacgctgttttcagcccgccagcgatagagcgattatcagggggtttgctgcaagaagtgatagtgacaaacacattacctgtagcagagaagaattacttcccgcagttaacaatattatcagtggctaatctcctgggtgagaccatttggcgtgtgcatgatgatagttccgtcagtagcattttcctttga

SEQ ID NO: 4 - protein, 313 aa, Arabidopsis thaliana
MVLKLFSGTANPALAQEIAWYMGLDLGKVNIKRFADGEIYVQLQESVRGCDVYLVQPTCTPTNENLMELLIMVDACRRASAKKVTAVIPYFGYARADRKTQGRESIAAKLVANLITEAGADRVLACDLHSGQSMGYFDIPVDHVYCQPVILDYLASKSIPSEDLVVVSPDVGGVARARAFAKKLSDAPLAIVDKRRSGHNVAEVMNLIGDVRGKVAIMVDDMIDTAGTIVKGAALLHQEGAREVYACCTHAVFSPPAIERLSGGLLQEVIVTNTLPVAEKNYFPQLTILSVANLLGETIWRVHDDSSVSSIFL

SEQ ID NO: 5 - DNA, 530 bp, Physcomitrella patens
  CDS (1)..(528)
cacgagttgtcgccgcacccttgccgccgaggatccttggcttcgccgcaagcgacgagaggcagcggccttttccttggagatagaaattggaggcaacggagaggaccgctagcgcgaccgaaatgtgcgctagcggattcctcagtatcctggaatggaaggccagtagtacctgttgtgccgaatcagaattggaatagcgtggctgggtcaaaagtgctaagggatactgtgcttcatgatatccgcttggagaatcgcctcaaaattttctctggcactgcaaataaggctctttcccaagaaatcgcgtactacatgggtctcgatttaggaaagataaccataaagcgcttcgcggacggagaaatctacgtacagctacaagagagtgttcgtggttgtgacgtgtttctagtgcaaccaacatgtccacctgcaaatgagaatctgatggagctcctcattatgatcgatgcatgccgccgagcttctgctaagaacataacagctgtgataccatactt

SEQ ID NO: 6 - protein, 176 aa, Physcomitrella patens
HELSPHPCRRGSLASPQATRGSGLFLGDRNWRQRRGPLARPKCALADSSVSWNGRPVVPVVPNQNWNSVAGSKVLRDTVLHDIRLENRLKIFSGTANKALSQEIAYYMGLDLGKITIKRFADGEIYVQLQESVRGCDVFLVQPTCPPANENLMELLIMIDACRRASAKNITAVIPY

(19) United States
(12) Patent Application Publication
(10) Pub. No.: US 2004/0241648 A1
(43) Pub. Date: Dec. 2, 2004

(54) Phosphoribosyl pyrophosphate synthetase 1as herbicidal target

(76) Inventors: Andreas Reindl, Birkenheide (DE); Jens Lerchl, Ladenburg (DE); Uwe Sonnewald, Quedlinburg (DE); Ralf Badur, Goslar (DE); Ralf Boldt, Quedlinburg (DE)

Correspondence Address: Keil & Weinkauf, 1101 Connecticut Avenue NW, Washington, DC, 20036, US

(21) Appl. No.: 10/220,154

(22) PCT Filed: Jan. 30, 2001
(86) PCT No.: PCT/EP01/00971

(30) Foreign Application Priority Data
Feb. 28, 2000 (DE) 10009277.2

(51) Int. Cl. C12Q 1/68; C07H 21/04

(52) U.S. Cl. 435/6; 536/23.2

(57) ABSTRACT

The present invention relates to the use of DNA sequences of plant origin encoding a polypeptide with phosphoribosyl-pyrophosphate synthetase 1 (E.C. 2.7.6.1) activity for the preparation of a test system for identifying phosphoribosyl-pyrophosphate synthetase 1 inhibitors. It was shown for the first time, using antisense technology, that phosphoribosyl-pyrophosphate synthetase 1 is a herbicidal [sic] target.

The present invention relates to the identification of plant phosphoribosyl-pyrophosphate synthetase 1 (E.C. 2.7.6.1) as novel target for herbicidal active ingredients. The present invention also relates to the use of the DNA sequence SEQ-ID No. 1, SEQ-ID No. 3 or SEQ-ID No. 5 or parts of derivatives thereof encoding a polypeptide with phosphoribosyl-pyrophosphate synthetase 1 activity for the generation of an assay system for identifying herbicidally active phosphoribosyl-pyrophosphate synthetase 1 inhibitors. The invention also relates to a method of or an assay system for identifying substances which inhibit the activity of the plant phosphoribosyl-pyrophosphate synthetase 1, and inhibitors of plant phosphoribosyl-pyrophosphate synthetase 1 identified using these methods or this assay system.

Plants are capable of synthesizing their cellular components from carbon dioxide, water and inorganic salts.

This process is only possible by exploiting biochemical reactions for the synthesis of organic substances. Nucleotides are synthesized de novo in plants. Being components of the nucleic acids, they are of particular importance. Covalently bonded, nucleotides activate carbohydrates for polysaccharide biosynthesis. They furthermore activate head groups for lipid biosynthesis. Nucleotides participate in virtually all metabolic pathways. Nucleoside triphosphates, in particular ATP, drive most of the energy-dependent reactions of the cell. Adenine nucleotides are additionally found as a component in essential factors such as coenzyme A, and nicotinamide and flavin coenzymes which participate in a large number of cellular reactions. The coupled hydrolysis of guanosin 5′-triphosphate (GTP) defines a direction of reaction for various cellular processes such as protein translation, microtubuli assembly, vesicular transport, signal transduction and cell division. Furthermore, nucleotides constitute the starting metabolites for the biosynthesis of methylxanthins such as caffeine and theobromine in the plant families of the Rubiaceae and Theaceae.

Since plants depend on an efficient nucleotide metabolism, it can be assumed that enzymes which participate in nucleotide biosynthesis are a suitable target protein for herbicides. Thus, there have already been described active ingredients which inhibit plant de novo purinebiosynthesis. An example which may be mentioned is the naturally occurring substance hydanthocidin, which, after phosphorylation in plants, inhibits adenylosuccinate synthetase (ASS), (Siehl et al., Plant Physiol. 110(1996), 753-758).

Phosphoribosyl-pyrophosphate (PRPP) is an essential unit in plant metabolism. It is required in the de-novo synthesis of purine and pyrimidine nucleotides, the pyridine nucleotide coenzyme NAD, and for the reutilization of purines, pyrimidines and pyridines which have already been synthesized. PRPP is furthermore required in the synthesis of histidine and tryptophane (see Krath and Hove-Jensen, Plant Physiology 119(1999), 497-505). The enzyme responsible for the synthesis of PRPP is PRPP synthetase, which converts ribose-5-phosphate and MgATP to AMP and PRPP. The phosphoribosyl-pyrophosphate synthetase of some organisms additionally require Mg²⁺. Eukaryotes frequently have more than one phosphoribosyl-pyrophosphate synthetase. Thus, for example, humans have three isoforms with redundant function. Plant phosphoribosyl-pyrophosphate synthetase, of which, for example, four isoforms are present in spinach, can be broken down into two classes and differ with regard to localization and function, or are subject to different development-dependent regulations of the plant cell. For example, phosphoribosyl-pyrophosphate synthetase 2 is imported into the chloroplasts (Krath and Hove-Jensen, Plant Physiology 119(1999), 497-505). This tallies with results where several enzymes of purine de-novo biosynthesis have been detected in the chloroplasts of Arabidopsis (Senecoff and Meager, Plant Physiology 102(1993), 387-399; Ito et al., Plant Mol Biol 26(1994), 529-533; Schnorr et al., Plant Journal 6(1994), 113-121). In yeast, it was demonstrated that the four phosphoribosyl-pyrophosphate synthetase isoforms have different functions (Carter et al., Yeast 10(1994), 1031-1044). In contrast to human phosphoribosyl-pyrophosphate synthetase, other higher eukaryotes and Saccharomyces cerevisiae, only little research has centered on plant phosphoribosyl-pyrophosphate synthetase. General methods of identifying inhibitors of the plant purine metabolism have been described in U.S. Pat. Nos. 5,780,253 and 5,780,254.

Genes which encode phosphoribosyl-pyrophosphate synthetase 1 have been isolated from various organisms. In the case of plants, full-length DNA sequences have been isolated from Arabidopsis thaliana (Accession No. X 83764) and tobacco (Ph.D. Dr. Badur, University of Göttingen, 1998).

The suitability of an enzyme as target for herbicides can be confirmed by reducing the enzyme activity, for example by means of antisense technology in transgenic plants. If the introduction of an antisense DNA for a certain gene into a plant causes reduced growth, this suggests that the enzyme whose activity is reduced is suitable as site of action for herbicidal active ingredients. For example, antisense inhibition of acetolactate synthase (ALS) in transgenic potato plants, like the treatment of control plants with ALS-inhibiting herbicides, leads to comparable phenotypes (Höfgen et al., Plant Physiology 107(1995), 469-477).

It is an object of the present invention to provide proof that phosphoribosyl-pyrophosphate synthetase 1 in plants is a suitable herbicidal [sic] target, and to generate an efficient and simple phosphoribosyl-pyrophosphate synthetase 1 assay system for carrying out inhibitor-enzyme binding studies.

We have found that this object is achieved by the isolation of genes which encode the plant enzyme phosphoribosyl-pyrophosphate synthetase 1, the generation of antisense constructs of plant phosphoribosyl-pyrophosphate synthetase 1 and its expression in plants, and the functional expression of plant phosphoribosyl-pyrophosphate synthetase 1 in bacterial or eukaryotic cells.

To achieve the object, Nicotiana tabacum and Arabidopsis thaliana cDNAs encoding plant phosphoribosyl-pyrophosphate synthetase 1 and a Physcomitrella patens partial cDNA were isolated and sequenced, see Example 1 and Example 6, sequence listing SEQ-ID No. 1, SEQ-ID No. 3 and SEQ-ID No. 5.

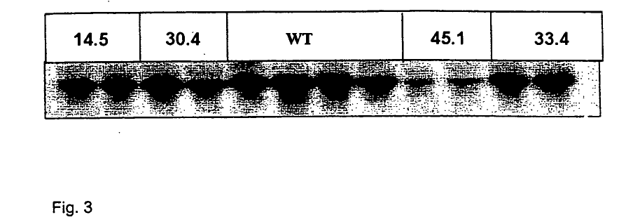

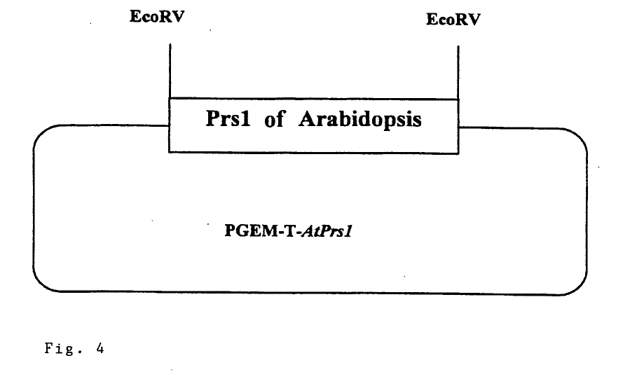

Arabidopsis thaliana plants and tobacco plants of the line Nicotiana tabacum cv. Samsun NN which carry a sense or antisense construct of phosphoribosyl-pyrophosphate synthetase 1 were characterized in greater detail. The antisense plants showed different degrees of retarded growth. The transgenic lines and the progeny of the 1^{st} and 2^{nd} generation showed a reduced growth in soil. Using Northern hybridization, it was detected that the RNA quantity of phosphoribosyl-pyrophosphate synthetase 1 was reduced in plants with reduced growth compared with the wild type, see Example 5 and FIG. 3. Furthermore, measurement of the enzyme activity detected that the amount of phosphoribosyl-pyrophosphate synthetase 1 activity was reduced in the transgenic antisense lines compared with wild-type plants. The expression level and the reduction in phosphoribosyl-pyrophosphate synthetase 1 activity correlate with the retarded growth. Even though it is highly probable that several isoforms of phosphoribosyl-pyrophosphate synthetase occur in plants, it has been found, surprisingly, that a reduced growth in the plant can be observed by introducing a phosphoribosyl-pyrophosphate synthetase antisense construct. This clear connection identifies phosphoribosyl-pyrophosphate synthetase 1 for the first time unambiguously as suitable target protein for herbicidal active ingredients.

Another subject-matter of the invention relates to methods of identifying plant phosphoribosyl-pyrophosphate synthetase 1 inhibitors by high-throughput methods. The invention therefore relates to the functional expression of plant phosphoribosyl-pyrophosphate synthetase 1, in particular tobacco and Arabidopsis thaliana phosphoribosyl-pyrophosphate synthetase 1 in suitable expression systems and to the use of the enzymes prepared thus in an in vitro assay system for measuring the phosphoribosyl-pyrophosphate synthetase 1 activity.

To be able to find efficient plant phosphoribosyl-pyrophosphate synthetase 1 inhibitors, it is necessary to provide suitable assay systems with which inhibitor-enzyme binding studies can be carried out. For this purpose, for example the cDNA sequence of phosphoribosyl-pyrophosphate synthetase 1 or suitable fragments of the cDNA sequence of tobacco or Arabidopsis thaliana phosphoribosyl-pyrophosphate synthetase 1 is cloned into an expression vector (pQE, Qiagen) and overexpressed in E. coli.

Alternatively, however, it is possible to express the expression cassette containing a DNA sequence, or DNA subsequence, of SEQ-ID No. 1 or SEQ-ID No. 3 or derivatives of these sequences for example in other bacteria, in yeasts, fungi, algae, plant cells, insect cells or mammalian cells.

Another subject-matter of the invention is the use of DNA sequences which are derived from SEQ-ID No. 1 or SEQ-ID No. 3 or which hybridize with one of these sequences and which encode a protein which has the biological activity of a phosphoribosyl-pyrophosphate synthetase 1.

The plant phosphoribosyl-pyrophosphate synthetase 1 protein which is expressed with the aid of an expression cassette is particularly suitable for identifying inhibitors which are specific for phosphoribosyl-pyrophosphate synthetase 1.

To this end, it is possible, for example, to employ the plant phosphoribosyl-pyrophosphate synthetase 1 in an enzyme assay in which the activity of the phosphoribosyl-pyrophosphate synthetase 1 is determined in the presence and absence of the active ingredient to be tested. By comparing the two activity measurements, a qualitative and quantitative statement can be made on the inhibitory behavior of the active ingredient to be tested, see Example 7.

The assay system according to the invention allows a large number of chemicals to be tested rapidly and simply for herbicidal properties. Using this method, substances with a potent action can be selected specifically and reproducibly from amongst a large number of substances, in order that further in-depth tests with which the skilled worker is familiar are carried out subsequently with these substances.

Another subject-matter of the invention is a method of identifying plant phosphoribosyl-pyrophosphate synthetase 1 inhibitors with a potentially herbicidal action by cloning the gene of a plant phosphoribosyl-pyrophosphate synthetase 1, overexpressing it in a suitable expression cassette—for example in insect cells—, disrupting the cells and employing the cell extract in an assay system for measuring the enzyme activity in the presence of low-molecular-weight chemicals, either directly or after concentration or isolation of the enzyme phosphoribosyl-pyrophosphate synthetase 1.

Another subject-matter of the invention is a method of identifying herbicidally active substances which inhibit the phosphoribosyl-pyrophosphate synthetase 1 activity in plants, which method comprises

a) the generation of transgenic plants, plant tissues or plant cells which contain an additional DNA sequence encoding an enzyme with phosphoribosyl-pyrophosphate synthetase 1 activity and which are capable of overexpressing an enzymatically active phosphoribosyl-pyrophosphate synthetase 1;

b) applying a substance to transgenic plants, plant cells, plant tissues or plant organs and to untransformed plants, plant cells, plant tissues or plant organs;

c) determining the growth or the survival capacity of the transgenic and the untransformed plants, plant cells, plant tissues or plant organs after application of the chemical substance; and

d) comparing the growth or the survival capacity of the transgenic and the untransformed plants, plant cells, plant tissues or plant organs after application of the chemical substance;

where suppression of the growth or the survival capacity of the untransformed plants, plant cells, plant tissues or plant organs—without substantial suppression of the growth or the survival capacity of the transgenic plants, plant cells, plant tissues or plant organs, however—confirms that the substance under b) has herbicidal activity and inhibits the phosphoribosyl-pyrophosphate synthetase 1 enzyme activity in plants.

Another subject-matter of the invention are herbicidally active compounds which can be identified with the above-described assay system.

Herbicidally active phosphoribosyl-pyrophosphate synthetase 1 inhibitors can be applied as defoliants, desiccants, haulm killers and, in particular, as weed killers. Weeds in the widest sense are to be understood as meaning all plants which grow in locations where they are undesired. Whether the active ingredients identified with the aid of the assay system according to the invention act as total or selective herbicides depends, inter alia, on the quantity applied.

Herbicidally active phosphoribosyl-pyrophosphate synthetase 1 inhibitors can be used, for example, against the following weeds:

Dicotyledonous weeds of the genera:

Sinapis, Lepidium, Gallium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds of the genera:

Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

Subject-matter of the invention is also the use of expression cassettes whose sequence encodes an Arabidopsis thaliana or Nicotiana tabacum phosphoribosyl-pyrophosphate synthetase 1 or its functional equivalent for the generation of an assay system for identifying herbicidally active compounds. The nucleic acid sequence may be, for example, a DNA or a cDNA sequence.

Such expression cassettes furthermore contain regulatory nucleic acid sequences which govern the expression of the encoding sequence in the host cell. In accordance with a preferred embodiment, an expression cassette according to the invention encompasses upstream, i.e. at the 5′ end of the encoding sequence, a promoter, and downstream, i.e. at the 3′ end, a polyadenylation signal and, if appropriate, other regulatory elements which are operatively linked to the encoding sequence for the phosphoribosyl-pyrophosphate synthetase 1 gene, which sequence lies between the promoter and the polyadenylation signal. Operative linkage is to be understood as meaning the sequential arrangement of promoter, encoding sequence, terminator and, if appropriate, other regulatory elements in such a manner that each of the regulatory elements can function as intended when the encoding sequence is expressed.

Such an expression cassette according to the invention is generated by fusing a suitable promoter with a suitable phosphoribosyl-pyrophosphate synthetase 1 DNA sequence and a polyadenylation signal using customary recombination and cloning techniques as they are described, for example, by T. Maniatis, E. F. Fritsch and J. Sambrook, Molecular Cloning: A Laboratory Manual, Cold Spring Harbor Laboratory, Cold Spring Harbor, N.Y. (1989) and by T. J. Silhavy, M. L. Berman and L. W. Enquist, Experiments with Gene Fusions, Cold Spring Harbor Laboratory, Cold Spring Harbor, N.Y. (1984) and by Ausubel, F. M. et al., Current Protocols in Molecular Biology, Greene Publishing Assoc. and Wiley-Interscience (1987).

Subject-matter of the invention is also the use of functionally equivalent DNA sequences which encode a phosphoribosyl-pyrophosphate synthetase 1 gene and which show a sequence homology with the DNA sequence SEQ-ID No. 1, SEQ-ID No. 3 or SEQ-ID No. 5 of 40 to 100% based on the total length of the DNA sequence.

Preferred subject-matter of the invention is the use of functionally equivalent DNA sequences which encode a phosphoribosyl-pyrophosphate synthetase 1 gene and which show a sequence homology with the DNA sequence SEQ-ID No. 1, SEQ-ID No. 3 or SEQ-ID No. 5 of 60 to 100%, based on the total length of the DNA sequence.

Particularly preferred subject-matter of the invention is the use of functionally equivalent DNA sequences which encode a phosphoribosyl-pyrophosphate synthetase 1 gene and which show a sequence homology with the DNA sequence SEQ-ID NO. 1, SEQ-ID NO. 3 or SEQ-ID No. 5 of 80 to 100%, based on the total length of the DNA sequence.

Functionally equivalent sequences which encode a phosphoribosyl-pyrophosphate synthetase 1 gene are in accordance with the invention those sequences which retain the desired functions, despite a deviating nucleotide sequence. Function equivalents thus encompass naturally occurring variants of the sequences described herein, but also artificial nucleotide sequences, for example those which have been obtained by chemical synthesis and which are adapted to suit the codon usage of a plant.

A functional equivalent is also to be understood as meaning in particular natural or artificial mutations of an originally isolated sequence which encodes a phosphoribosyl-pyrophosphate synthetase 1 and which continues to show the desired function. Mutations encompass substitutions, additions, deletions, exchanges or insertions of one or more nucleotide residues. Thus, the present invention for example also extends to those nucleotide sequences which are obtained by modifying the nucleotide sequence. The target of such a modification can be, for example, the further delimitation of the encoding sequence contained therein or else, for example, the introduction of further restriction enzyme cleavage sites.

Functional equivalents are also those variants whose function is reduced or increased compared with the starting gene or starting fragment.

In addition, the expression cassette according to the invention can also be employed for the transformation of bacteria, cyanobacteria, yeasts, filamentous fungi and algae, with the purpose of producing sufficient amounts of the enzyme phosphoribosyl-pyrophosphate synthetase 1.

Another subject-matter of the invention is the use of a Nicotiana tabacum or Arabidopsis thaliana protein characterized by the amino acid sequence SEQ-ID NO. 2, SEQ-ID No. 4 or derivatives or parts of this protein with phosphoribosyl-pyrophosphate synthetase 1 activity for the generation of an assay system for identifying herbicidally active compounds.

Subject-matter of the invention is also the use of plant proteins with phosphoribosyl-pyrophosphate synthetase 1 activity with an amino acid sequence homology to the Nicotiana tabacum, Arabidopsis thaliana or Physcomitrella patens phosphoribosyl-pyrophosphate synthetase 1 with the [lacuna] SEQ-ID NO. 2, SEQ-ID NO. 4 or SEQ-ID No. 6 of 20-100% identity for the generation of an assay system for identifying herbicidally active compounds.

Also preferred is the use of plant proteins with phosphoribosyl-pyrophosphate synthetase 1 activity with an amino acid sequence homology to the Nicotiana tabacum, Arabidopsis thaliana or Physcomitrella patens phosphoribosyl-pyrophosphate synthetase 1 with the sequences SEQ-ID NO. 2, SEQ-ID NO. 4 or SEQ-ID No. 6 of 50-100% identity for the generation of an assay system for identifying herbicidally active compounds.

Particularly preferred is the use of plant proteins with phosphoribosyl-pyrophosphate synthetase 1 activity with an amino acid sequence homology to the Nicotiana tabacum, Arabidopsis thaliana or Physcomitrella patens phosphoribosyl-pyrophosphate synthetase 1 with the sequences SEQ-ID NO. 2, SEQ-ID NO. 4 or SEQ-ID No. 6 of 80-100% identity for the generation of an assay system for identifying herbicidally active compounds.

Overexpression, in a plant, of the gene sequence SEQ-ID NO. 1 or SEQ-ID NO. 3, which encodes a phosphoribosyl-pyrophosphate synthetase 1, results in increased resistance to phosphoribosyl-pyrophosphate synthetase 1 inhibitors. The transgenic plants generated thus are also subject-matter of the invention.

Expressional efficacy of the recombinantly expressed phosphoribosyl-pyrophosphate synthetase 1 gene can be determined, for example, in vitro by shoot-meristem propagation or a germination test. Moreover, the expression of the phosphoribosyl-pyrophosphate synthetase 1 gene which has been altered in terms of type and level, and its effect on the resistance to phosphoribosyl-pyrophosphate synthetase 1 inhibitors, can be tested in greenhouse experiments using test plants.

Subject-matter of the invention are also transgenic plants, transformed with an expression cassette according to the invention containing the DNA sequence SEQ-ID No. 1 or SEQ-ID No. 3, which have been made tolerant to phosphoribosyl-pyrophosphate synthetase 1 inhibitors by additionally expressing the DNA sequence SEQ-ID No. 1 or SEQ-ID No. 3, and transgenic cells, tissues, organs and propagation material of such plants. Especially preferred are transgenic crop plants such as, for example, barley, wheat, rye, maize, soya beans, rice, cotton, sugarbeet, canola, sunflowers, flax, hemp, potatoes, tobacco, tomatoes, oilseed rape, alfalfa, lettuce and the various tree, nut and grapevine species, and legumes.

Especially preferred are sequences which ensure a targeting into the apoplasts, into plastids, into the vacuole, the mitochondrion, the endoplasmatic reticulum (ER) or which, owing to a lack of suitable operative sequences, ensure that the expression products remain in the compartment in which it has been formed, the cytosol (Kermode, Crit. Rev. Plant Sci. 15, 4 (1996), 285-423).

By way of example, the plant expression cassette can be incorporated into the plant transformation vector pBinAR.

A suitable promoter of the expression cassette is, in principle, any promoter which is capable of governing the expression of foreign genes in plants. It is preferred to use, in particular, a plant promoter or a promoter derived from a plant virus. Especially preferred is the cauliflower mosaic virus CaMV 35S promoter (Franck et al., Cell 21(1980) , 285-294). This promoter contains different recognition sequences for transcriptional effectors which, in their entirety, lead to permanent and constitutive expression of the gene which has been introduced (Benfey et al., EMBO J., 8 (1989), 2195-2202).

The expression cassette may also contain a chemically inducible promoter which allows expression of the exogenous phosphoribosyl-pyrophosphate synthetase 1 gene in plants to be governed at a particular point in time. Such promoters which are described in the literature and which can be used are, inter alia, for example the PRP1 promoter (Ward et al., Plant. Mol. Biol. 22(1993), 361-366), a salicylic acid-inducible promoter (WO 95/19443), a benzenesulfonamide-inducible promoter (EP 388186), a tetracyclin-inducible promoter (Gatz et al., Plant J. 2(1992), 397-404), an abscisic acid-inducible promoter (EP0335528), or an ethanol- or cyclohexanone-inducible promoter (WO 93/21334).

Especially preferred promoters are furthermore those which ensure expression in tissues or plant organs in which the biosynthesis of purines or their precursors take place. Promoters which ensure leaf-specific expression must be mentioned in particular. Promoters which must be mentioned are the potato cytosolic FBPase or the potato ST-LSI promoter (Stockhaus et al., EMBO J., 8 (1989) 2445[sic]-245).

A foreign protein can be expressed stably in the seeds of transgenic tobacco plants to an extent of 0.67% of the total soluble seed protein with the aid of a seed-specific promoter (Fiedler and Conrad, Bio/Technology 10 (1995), 1090-1094). The expression cassette according to the invention can therefore contain, for example, a seed-specific promoter (preferably the phasolin promoter, the USP promoter or the LEB4 promoter), the LEB4 signal peptide, the gene to be expressed and an ER-retention signal.

The inserted nucleotide sequence encoding a phosphoribosyl-pyrophosphate synthetase 1 can be produced synthetically or obtained naturally or contain a mixture of synthetic and natural DNA components. In general, synthetic nucleotide sequences are generated with codons which are preferred by plants. These codons which are preferred by plants can be determined from codons with the highest protein frequency expressed in the plant species of highest interest. When preparing an expression cassette, a variety of DNA fragments may be manipulated in order to obtain a nucleotide sequence which expediently reads in the correct direction and which is equipped with a correct reading frame. Adapters or linkers can be added to the fragments in order to link the DNA fragments to each other.

Other suitable DNA sequences are artificial DNA sequences as long as they mediate the desired property of expressing the phosphoribosyl-pyrophosphate synthetase 1 gene. Such artificial DNA sequences can be determined for example by backtranslating proteins which have phosphoribosyl-pyrophosphate synthetase 1 activity, or they can be determined by in-vitro selection. Especially suitable are encoding DNA sequences which have been obtained by backtranslating a polypeptide sequence in accordance with the host-plant-specific codon usage. The specific codon usage can be determined readily by a skilled worker familiar with the methods of plant genetics by means of computer evaluations of other, known genes of the plant to be transformed.

Other suitable equivalent nucleic acid sequences according to the invention which must be mentioned are sequences which encode fusion proteins, the component of the fusion protein being a plant phosphoribosyl-pyrophosphate synthetase 1 polypeptide or a functionally equivalent part thereof. The second part of the fusion protein can be, for example, another polypeptide with enzymatic activity or an antigenic polypeptide sequence, with the aid of which detection of phosphoribosyl-pyrophosphate synthetase 1 expression is possible (for example myc-tag or his-tag). However, it is preferably a regulatory protein sequence such as, for example, a signal or transit peptide, which leads the phosphoribosyl-pyrophosphate synthetase 1 protein to the desired site of action.

The promoter and terminator regions should expediently be provided, in the direction of transcription, with a linker or polylinker containing one or more restriction sites for insertion of this sequence. As a rule, the linker has 1 to 10, in most cases 1 to 8, preferably 2 to 6, restriction sites. In general, the linker within the regulatory regions has a size of less than 100 bp, frequently less than 60 bp, but at least 5 bp. The promoter according to the invention may be native, or homologous, or else foreign, or heterologous, to the host plant. The expression cassette according to the invention comprises, in the 5′-3′ direction of transcription, the promoter according to the invention, any sequence, and a region for transcriptional termination. Various termination regions can be exchanged for each other as desired.

Manipulations which provide suitable restriction cleavage sites or which eliminate excess DNA or restriction cleavage sites may also be employed. In-vitro mutagenesis, primer repair, restriction or ligation can be used in cases where insertions, deletions or substitutions such as, for example, transitions and transversions are suitable.

Complementary ends of the fragments may be provided for ligation in the case of suitable manipulations such as, for example, restriction, chewing-back or filling in overhangs for blunt ends.

Preferred polyadenylation signals are plant polyadenylation signals, preferably those which correspond essentially to Agrobacterium tumefaciens T-DNA polyadenylation signals, in particular those of gene 3 of the T-DNA (octopine synthase) of the Ti plasmid pTiACH5 (Gielen et al., EMBO J., 3 (1984), 835), or functional equivalents.

To transform a host plant with a DNA encoding a phosphoribosyl-pyrophosphate synthetase 1, an expression cassette according to the invention is incorporated, as insertion, into a recombinant vector whose vector DNA contains additional functional regulatory signals, for example sequences for replication or integration. Suitable vectors are described, inter alia, in “Methods in Plant Molecular Biology and Biotechnology” (CRC Press, Chapters 6/7, 71-119).

The transfer of foreign genes into the genone of a plant is termed transformation. It exploits the above-described methods of transforming and regenerating plants from plant tissues or plant cells for transient or stable transformation. Suitable methods are the protoplast transformation by polyethylene glycol-induced DNA uptake, the biolistic method using the gene gun, electroporation, the incubation of dry embryos in DNA-containing solution, microinjection, and the agrobacterium-mediated gene transfer. The abovementioned methods are described, for example, by B. Jenes et al., Techniques for Gene Transfer, in: Transgenic Plants, Vol. 1, Engineering and Utilization, edited by S. D. Kung and R. Wu, Academic Press (1993), 128-143, and by Potrykus, Annu. Rev. Plant Physiol. Plant Molec. Biol. 42 (1991), 205-225. The construct to be expressed is preferably cloned into a vector which is suitable for the transformation of Agrobacterium tumefaciens, for example pBin19 (Bevan et al., Nucl. Acids Res. 12 (1984), 8711).

Agrobacteria transformed with an expression cassette according to the invention can equally be used in a known manner for transforming plants, in particular crop plants such as cereals, maize, soya beans, rice, cotton, sugarbeet, canola, sunflowers, flax, hemp, potatoes, tobacco, tomatoes, oilseed rape, alfalfa, lettuce and the various tree, nut and grapevine species and legumes, for example by bathing scarified leaves or leaf sections into an agrobacterial suspension and subsequently growing them in suitable media.

The purine biosynthesis site is generally the leaf tissue, so that leaf-specific expression of the phosphoribosyl-pyrophosphate synthetase 1 gene is meaningful. However, it is obvious that purine biosynthesis need not be limited to the leaf tissue, but may also take place in all other remaining parts of the plant in a tissue-specific fashion, for example in fatty seeds.

In addition, constitutive expression of the exogenous phosphoribosyl-pyrophosphate synthetase 1 gene is advantageous. On the other hand, inducible expression may also be desirable.

Using the recombination and cloning techniques cited above, the expression cassettes according to the invention can be cloned into suitable vectors which allow them to be multiplied, for example in E. coli. Suitable cloning vectors are, inter alia, pBR332, pUC series, M13mp series and pACYC184. Especially suitable are binary vectors, which are capable of replication both in E. coli and in agrobacteria.

Another subject-matter of the invention relates to the use of an expression cassette according to the invention for transforming plants, plant cells, plant tissues or plant organs. The preferred purpose of the use is to increase the phosphoribosyl-pyrophosphate synthetase 1 content in the plant.

Depending on the choice of the promoter, expression may take place specifically in the leaves, in the seeds or in other parts of the plant. Such transgenic plants and their propagation material and their plant cells, plant tissue or plant organs are another subject-matter of the present invention.

The invention will now be illustrated by the examples which follow, without being limited thereto.

Recombinant methods on which the use examples are based:

General Cloning Methods

Cloning methods such as restriction cleavages, DNA isolation, agarose gel electrophoresis, purification of DNA fragments, transfer of nucleic acids to nitrocellulose and nylon membranes, linking of DNA fragments, transformation of E. coli cells, bacterial cultures, and sequence analysis of recombinant DNA were carried out as described by Sambrook et al., Cold Spring Harbor Laboratory Press (1989); ISBN 0-87969-309-6. The transformation of agrobacterium tumefaciens was carried out following the method of Höfgen and Willmitzer (Nucl. Acid Res. 16(1988), 9877). The agrobacteria were grown in YEB medium (Vervliet et al., Gen. Virol. 26(1975), 33).

Sequence Analysis of Recombinant DNA

Recombinant DNA molecules were sequenced using an ABI laser fluorescence DNA sequencer, following the method of Sanger (Sanger et al., Proc. Natl. Acad. Sci. USA, 74 (1977), 5463-5467). Fragments resulting from a polymerase chain reaction were sequenced and checked to avoid polymerase errors in constructs to be expressed.

Analysis of Total RNA from Plant Tissues

Plant total RNA was isolated by the method of Logemann et al., Analytical Biochem. 163(1987), 16) and separated in formaldehyde-containing agarose gels (Lehrach et al., Biochem. 16 (1977), 4743). Capillary transfer to nylon membranes (Gene Screen, NEN) was done in 20×SSC (1.5 M NaCl, 150 mM sodium citrate) overnight. After two hours of prehybridization in hybridization buffer (500 mM sodium phosphate (pH 7.2), 7% SDS, 0.5% bovine serum albumin, 1 mM EDTA), hybridization was carried out for 16 hours at 65° C using a radiolabelled NtPrs1 probe. The filters were subsequently washed under the following conditions: 10 minutes at 65° C. in 6×SSC, 0.1% SDS and 5 minutes at 65 C [sic] in 4×SSC, 0.1% SDS.

Unless otherwise specified, the chemicals used were analytical grade and obtained from Fluka (Neu-Ulm), Merck (Darmstadt), Roth (Karlsruhe), Serva (Heidelberg) and Sigma (Deisenhofen). Solutions were made with purified, pyrogen-free water, termed H₂O below, from a Milli-Q water purification system (Millipore, Eschborn). Restriction endonucleases, DNA-modifying enzymes and molecular biology kits were obtained from AGS (Heidelberg), Amersham (Braunschweig), Biometra (Göttingen), Roche (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Heidelberg). Unless otherwise specified, they were used according to the manufacturer's instructions.

E. coli (XL-1 Blue) bacteria were obtained from Stratagene. The agrobacterial strain employed for the plant transformation (C58C1 with the plasmid pGV 3850kan) was described by Debleare et al., Nucl. Acid Res. 13(1985), 4777).

### EXAMPLE 1

Isolation of cDNA clones encoding the Nicotiana tabacum and Arabidopsis thaliana 5-phosphoribosyl-1-pyrophosphate synthetase 1 (Prs1)

Using a cDNA sequence encoding Arabidopsis thaliana Prs1 (Accession Number X83764; Krath and Hove-Jensen, 1995) oligonucleotides of the following sequence were deduced:

RBPRPP3 5′-aag aat tcg gat cca cca tgg tct tga agt tgt tct ctg gta ctg c-3′

RBPRPP4 5′-aag aat tcg gat cct caa agg aaa atg cta ctg ac-3′

Using these oligonucleotides, a cDNA fragment of Arabidopsis thaliana (var. Landsberg erecta) leaf cDNA was amplified (35 cycles, 30 sec at 94° C., 45 sec at 45° C., 2 min at 72° C.) and subsequently blunt-cloned into a pGEM-T vector (Promega) via EcoRV. The identity of the Arabidopsis Prs1 cDNA clone was identified by sequencing, see SEQ-ID No. 3.

This clone was employed for screening a source leaf cDNA library from Nicotiana tabacum variety Samsun NN in λZAPII. The cDNA library was plated at a titer of 2.5×10⁵ plaque-forming units and analyzed with the aid of the plaque screening method (T. Maniatis, E. F. Fritsch and J. Sambrook, Molecular Cloning: A Laboratory Manual, Cold Spring Harbor Laboratory, Cold Spring Harbor, N.Y. 1989). 12 phage populations were isolated and used to carry out the second screening step, resulting in the isolation of genetically uniform phage populations which were used for in-vivo excision. Restriction analysis failed to find any differences between the cDNA clones, and four clones with the largest insertions were selected for sequencing. The sequence data show that they are cDNA clones encoding 5-phosphoribosyl-1-pyrophosphate synthetase.

The cDNA clone NtPrs1—see SEQ-ID No. 1—is 1251 bp long and has a start codon in position 21 and a termination codon TAG in position 1089. The open reading frame encodes a protein encompassing 356 amino acids and having a molecular mass of 38.3 kDa. The 152 bp 3′-untranslated region contains no polyadenylation signal. When analyzing the protein sequence deduced from the cDNA clone NtPrs1—see SEQ-ID No. 2—computer analysis identified a chloroplast transit peptide within the first 36 amino acids.

### EXAMPLE 2

### Generation of Binary Vectors for Transforming Tobacco Plants

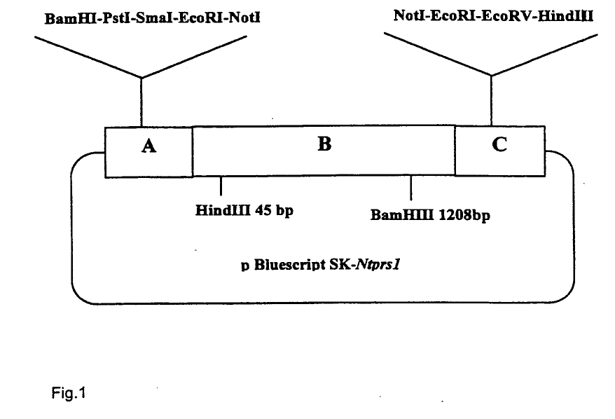

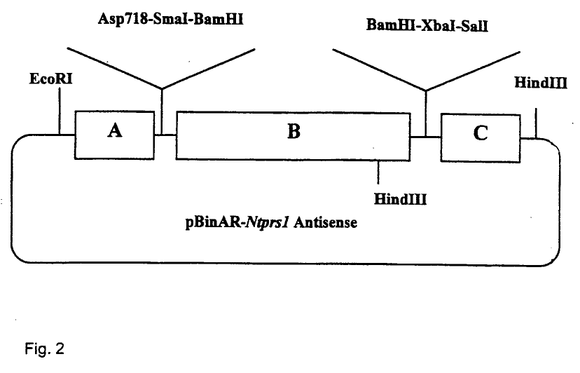

To generate plasmid pBinAR-NtPrs1 in antisense orientation, the binary vector pBinAR (Höfgen and Willmitzer, Plant Science 66(1990), 221-230) was cleaved in the polylinker using BamHI. A 1226 bp NtPrs1 cDNA fragment (SEQ-ID No. 1) was isolated from plasmid pBluescript SK-NtPrs1 (FIG. 1) via the BamHI cleavage site in the polylinker of the pBluescript SK plasmid and a BamHI cleavage site in the NtPrs1 cDNA (1208 bp). This fragment was ligated into the BamHI-cleaved vector pBinAR. The orientation of the ligated NtPrs1 cDNA fragment was checked by restriction with HindIII. The resulting 300 bp HindIII fragment confirmed the antisense orientation. Plasmid pBinAR-NtPrs1-antisense (FIG. 2) is composed of 3 fragments A, B and C. Fragment A contains, as constituent of the vector pBinAR, the cauliflower mosaic virus (CaMV) 35S promoter, which leads to constitutive expression in transgenic plants. This fragment encompasses nucleotides 6909 to 7437 of CaMV (Franck, Cell 21(1980), 285). Fragment B contains a 1226 bp NtPrs1 cDNA fragment, which was isolated from plasmid pBluescript SK-NtPrs1 as BamHI fragment and cloned into the BamHI site of the polylinker of vector pBinAR. Fragment C contains the polyadenylation signal of gene 3 (octopine synthase, OCS) of the T-DNA of the Ti plasmid pTiACH5 (Gielen et al., EMBO J. 3(1984), 835), which encompasses the nucleotides 11749-11939.

As shown in FIG. 1, the generation of the cDNA library included providing individual fragments with EcoRI/NotI linkers and subsequently cloning them into the EcoRI restriction sites of the pBluescript polylinker. Accordingly, the NotI cleavage sites are not an original component of the pBluescript polylinker.

A Multiple cloning site of plasmid pBluescript SK

B NtPrs1 cDNA 1251 bp

C Multiple cloning site of plasmid pBluescript SK

FIG. 2 shows the expression cassette for expressing the NtPrs1 cDNA (1226 bp) in antisense orientation in transgenic tobacco plants

A: Cauliflower mosaic virus (CaMV) 35S promoter, 529 bp, nucleotides 6909 to 7437 of CaMV.

B: NtPrs1 cDNA (1226 bp) in antisense orientation, which was isolated from plasmid pBluescript SK-NtPrs1 as BamHI fragment and encompasses the bases 1-1208 of the NtPrs1 coding region.

C: Polyadenylation signal of gene 3 (octopine synthase, OCS) of the T-DNA of the Ti plasmid pTiACH5 (192 bp).

### EXAMPLE 3

### Transformation of Tobacco Plants by Means of Agrobacterium tumefaciens

Tobacco plants were transformed by the method of Rosahl, S., Schell, J. and Willmitzer, L., EMBO J. 6(1987), 1155-1159. Plasmid pBinAR-NtPrs1 antisense (FIG. 2) was transformed into Agrobacterium tumefaciens C58C1 with plasmid pGV 3850kan (Debleare et al., Nucl. Acid Res. 13(1985), 4777). To transform tobacco plants (Nicotiana tabacum cv. Samsun NN), 10 ml of an overnight culture of a positively transformed agrobacterial colony in YEB medium (Vervliet et al., Gen. Virol. 26 (1975), 33) were used. Leaf disks of sterile plants (approx. 1 cm² each) were incubated in this solution for 5-10 minutes in a petri dish. This was followed by two days' incubation in the dark at 25° C. on MS medium (Murashige-Skoog medium, Murashige and Skoog, Plant Physiology, 15(1962), 473) with 0.8% BiTek™ agar (DIFCO Laboratories). After two days, cultivation was continued at 16 hours light/8 hours dark and continued in a weakly rhythm on MS medium with 500 mg/l Claforan (Cefotaxime-sodium), 100 mg/l kanamycin, 1 mg/l benzylaminopurinee (BAP), 0.2 mg/l naphthylacetic acid and 1.6 g/l glucose. Growing shoots were transferred to MS medium with 2% sucrose, 250 mg/l Claforan and 0.8% BiTek™ agar. After rooting, the shoots were transferred into soil, grown for two weeks in a controlled-environment cabinet with 16 hours light and 8 hours darkness, then repotted into larger pots and transferred into the greenhouse.

### EXAMPLE 4

### Analysis of the Transgenic Plants

Transgenic plants which are transformed with the construct pBinAR-NtPrs1 antisense are characterized by large chlorotic zones in the leaves and by strong bleaching of the leaves compared to untransformed wild-type plants. In general, the transgenic pBinAR-NtPrs1-antisense plants include plants whose growth is severely adversely affected.

This data constitutes a direct relationship between reduced phosphoribosyl-pyrophosphate synthetase 1 expression and reduced growth in tobacco plants and therefore identifies phosphoribosyl-pyrophosphate synthetase 1 as suitable target protein for herbicidal active ingredients.

### EXAMPLE 5

### Verification of the NtPrs1 Antisense Gene Expression by Northern Analysis

RNA analysis was done by strand-specific labeling of an NtPrs1 cDNA fragment. In order to carry out strand-specific radiolabeling, the plasmid pBluescript SK-NtPrs1 was cleaved with EcoRI, and a 1251 bp fragment which has the coding region of the NtPrs1 gene was isolated. A 3′ oligonucleotide with the following sequence: 5′CTT CAA GTT CCA GAC AAC AGT GTC-3′ was employed in the reaction. The kit used in the reaction was one by Finnzymes Oy, whose instructions were used. The reaction mixture contained approx. 10 ng fragment DNA, 0.1 mM of the oligonucleotide, 5 ml 10× buffer, 1.5 mM MgCl₂, 5 mM deoxynucleotides (dGTP, DATP, dTTP), 50 mCi a-³²P-dCTP (Amersham) and 1 ml DyNazyme polymerase. The amplification conditions were chosen as follows:

Denaturation temperature: 95° C., 5 sec

Annealing temperature: 45° C., 30 sec

Elongation temperature: 72° C., 1 min

Number of cycles: 40

To carry out the Northern analysis, total RNA was isolated from sink leaves approx. 0.5-1.0 cm in size, 20 μg of RNA were separated in a formaldehyde-containing agarose gel, and the fragments were transferred to a nylon membrane by capillary blotting. The nylon membrane was hybridized with the strand-specifically-labeled NtPrs1 gene probe. FIG. 3 shows the hybridization signals for wild-type (WT) and transgenic plants.

The hybridization signals were subsequently quantified by means of a phospho-imager. In accordance with the plants' phenotype, transcript accumulation of NtPrs1 synthetase mRNA is dramatically reduced in line 45-1. Plants 33.4, 14.5 and 30.4 only show reduced mRNA accumulation and, analogously thereto, a less clearly pronounced growth phenotype.

### EXAMPLE 6

### Generation of a cDNA Library from Physcomitrella patens

Plants of the species Physcomitrella patens (Hedw.) B.S.G. from the public collection of the Genetics Department, University of Hamburg, were used for the present work. The strain used, 16/14, had been collected by H. L. K. Whitehouse in Gransden Wood, Huntingdonshire (England) and subcultured via spores by the method of Engel (Am. J. Bot. 55(1968), 438-446). The proliferation of the moss plants was carried out via spores and regeneration of gametophores. The protonemal tissue developing from the haploid spore consists of the high-chloroplast chloronema and the low-chloroplast caulonema, on which buds form after approx. 12 days. These buds grow into gametophores and contain antheridia and archegonia. Fertilization results in the diploid sporophyte, short seta and spore capsule in which the maturing spores develop.

To generate a cDNA library, the first-strand synthesis was carried out by means of murine leukemia virus reverse transcriptase (Roche, Mannheim, Germany) and oligo-d(T) primers, starting from polyA+ RNA isolated from 9-day-old protonema. The second-strand synthesis was carried out by incubation with DNA polymerase I, Klenow enzyme, RNase H digest at 12C ° C. (2 h), 16° C, (1 h) and 22° C. (1 h). The reaction was stopped by incubation at 65° C. (10 min) and transferred to ice. Double-stranded DNA was filled up by means of T4 DNA polymerase (Roche, Mannheim) at 37° C. (30 min), and nucleotides were separated off by phenol/chloroform extraction and chromatography over Sephadex G50. EcoRI adapters (Pharmacia, Freiburg, Germany) were ligated onto the cDNA ends by means of T4 DNA ligase (Roche, 12° C., 16h) and phosphorylated by means of polynucleotide kinase (Roche, 37° C., 30 min). The DNA was separated by means of gel electrophoresis and fragments of greater than 300 base pairs were isolated by means of phenol extraction and concentrated using Elutip-D columns (Schleicher and Schuell, Dassel, Germany). The resulting double-stranded DNA was ligated into lambda ZAPII, using the Gigapack Gold Kit (Stratagene, Amsterdam, Netherlands) following the manufacturer's instructions. In-vivo excision gave plasmid DNA which was used for transforming E. coli XLI blue bacteria. Clones from single colonies were grown in liquid culture, and plasmid DNA was isolated and employed for sequence reactions. An EST encoding 5-phosphoribosyl-1-pyrophosphate synthesis [sic] was identified by homology comparison, see SEQ-ID No. 5.

### EXAMPLE 7

### 5-Phosphoribosyl-1-pyrophosphate Synthetase 1 Assay System

To obtain active Arabidopsis thaliana 5-phosphoribosyl-1-pyrophosphate synthetase 1 for high-throughput screening methods, the Arabidopsis thaliana Prs1 fragment is obtained from vector pGEM-T AtPrs1 (FIG. 2) using the restriction enzymes BamHI and EcoRI (cleavage sites inserted via primers RBPRPP3 and RBPRPP4) and cloned into the identically cleaved transfer vector pFastBacHTb (GibcoBRL). The resulting construct pFASTBAC HTb-AtPrs1 is used in accordance with the manufacturer's instructions (GibcoBRL) for generating recombinant Baculovirus. This virus is employed in accordance with the manufacturer's instructions (GibcoBRL) for infecting Sf21 insect cells in order to generate active 5-phosphoribosyl-1-pyrophosphate synthetase 1 enzyme. The specific enzymatic phosphoribosyl-pyrophosphate synthetase 1 activity is measured photometrically after the cells have been sonicated. The following components are employed per 90 μl batch:

16.4 mM NaH₂P buffer pH 7.8; 0.3 mM phosphoenolpyruvate (Sigma P-71279); 0.3 mM MgCl₂; 0.15 mM ATP (Sigma A-3377); 0.06 mM NADH (Sigma N-8129); 0.3 U pyruvate kinase (Sigma P-9136); 0.2 U myokinase (Sigma M-5520); 0.3 units L-lactate dehydrogenase (Böhringer/Roche 127230); for the inhibitor assay 0.5-5 mM cordycepin 5′-triphosphate (Sigma C-9137); 64 mM ribose-5-phosphate (SIGMA R-7750).

After the cells had been sonicated in lysis buffer (50 mM NaH₂PO₄, 300 mM NaCl, pH 7.8), 18 μg of total protein are employed. Further purification can be effected in accordance with the manufacturer's instructions (Qiagen) via affinity chromatography of the his-tags which had been introduced by the vector. To change the buffer of the protein, PD-10 columns (Pharmacia) and standard concentration methods such as, for example, ammonium sulfate precipitation are employed. What is measured is the drop in NADH at 340 nm. It was demonstrated that cordycepin-5′-triphosphate, an inhibitor of the 5-phosphoribosyl-1-pyrophosphate synthetase of other higher eukaryotes, also inhibits the enzymatic activity of the plant 5-phosphoribosyl-1-pyrophosphate synthetase 1.

1. The use of a DNA sequence SEQ-ID No.1, SEQ-ID No.3 or SEQ-ID No.5 with the coding region of a plant phosphoribosyl-pyrophosphate synthetase 1, for the preparation of an assay system for identifying plant phosphoribosyl-pyrophosphate synthetase 1 inhibitors.

2. The use of a DNA sequence as claimed in claim 1, which hybridizes with the DNA sequence SEQ-ID No.1, SEQ-ID No.3 or SEQ-ID No.5 or parts thereof or derivatives which are derived from said sequences by insertion, deletion or substitution and which encodes a protein which has the bioactivity of plant phosphoribosyl-pyrophosphate synthetase 1.

3. The use of a DNA sequence as claimed in claim 1 for introduction into pro- or eukaryotic cells, this sequence optionally being linked to control elements which ensure transcription and translation in the cells and leading to the expression of a translatable mRNA which causes the synthesis of a plant phosphoribosyl-pyrophosphate synthetase 1.

4. The use a protein pyrophosphate synthetase 1 activity for the preparation of an assay system for identifying substances which inhibit the plant phosphoribosyl-pyrophosphate synthetase 1.

5. The use of a protein with phosphoribosyl-pyrophosphate synthetase 1 activity as claimed in claim 4, wherein the protein comprises the amino acid sequence shown in SEQ-ID No. 2, SEQ-ID No. 4 or SEQ-ID No. 6.

6. The use of a protein with phosphoribosyl-pyrophosphate synthetase 1 activity, comprising an amino acid subsequence of at least 100 amino acids from SEQ-ID No. 2 or SEQ-ID No. 4 as set forth in claim 5.

7. A method of identifying substances which inhibit the activity of the plant phosphoribosyl-pyrophosphate synthetase 1, which comprises generating, in a first step, phosphoribosyl-pyrophosphate synthetase I by using a DNA sequence as set forth in claim 1, and, in a second step, measuring the activity of the plant phosphoribosyl-pyrophosphate synthetase 1 in the presence of an assay substance.

8. A method as claimed in claim 7, wherein the plant phosphoribosyl-pyrophosphate synthetase 1 [lacuna] is measured in a high-throughput screening (HTS) step.

9. A method of identifying herbicidally active substances which inhibit the pyrophosphate synthetase 1 [lacuna] in plants, which method comprises
a) the generation of transgenic plants, plant tissues or plant cells which contain an additional DNA sequence encoding an enzyme with phosphoribosyl-pyrophosphate synthetase 1 activity and which are capable of overexpressing an enzymatically active phosphoribosyl-pyrophosphate synthetase 1;
b) applying a substance to transgenic plant cells, plant tissues or plant and to untransformed plants, plant tissues or plant organs;
c) determining the growth or the survival capacity of the transgenic and the untransformed plants, plant cells, plant tissues or plant organs after application of the chemical substance; and
d) comparing the growth or the survival capacity of the transgenic and the untransformed plants, plant cells, plant tissues or plant organs after applying the chemical substance;
where suppression of the growth or the survival capacity of the untransformed plants, plant cells, plant tissues or plant organs—without substantial suppression of the growth or the survival capacity of the transgenic plants, plant cells, plant tissues or plant organs, however—confirms that the substance under b) has herbicidal activity and inhibits the phosphoribosyl-pyrophosphate synthetase 1 enzyme activity in plants.

10. An assay system based on the expression of a DNA sequence SEQ-ID No. 1, SEQ-ID No. 3 or SEQ-ID No. 5 or parts thereof or derivatives as set forth in claim 1, for identifying plant phosphoribosyl-pyrophosphate synthetase 1 inhibitors.

11. An assay system as claimed in claim 10 for identifying plant phosphoribosyl-pyrophosphate synthetase 1 inhibitors, which comprises incubating the enzyme with a test substrate to be studied and, after a suitable reaction time, determining the enzymatic activity of the enzyme compared with the activity of the uninhibited enzyme.

12. A plant phosphoribosyl-pyrophosphate synthetase 1 inhibitor.

13. A plant phosphoribosyl-pyrophosphate synthetase 1 inhibitor, identified using an assay system as claimed in claim 10.

14. An inhibitor identified as claimed in claim 12 for use as herbicide.
